(19) United States
(12) Patent Application Publication
(10) Pub. No.: US 2020/0069322 A1
(43) Pub. Date: Mar. 5, 2020

(54) A SURGICAL DEVICE, A METHOD FOR ASSEMBLING AND A METHOD FOR De-ASSEMBLING

(71) Applicant: Surge-on Medical B.V., Delft (NL)

(72) Inventor: Tim HOREMAN, Leiderdorp (NL)

(73) Assignee: Surge-on Medical B.V., Delft (NL)

(21) Appl. No.: 16/467,682

(22) PCT Filed: Dec. 8, 2017
(86) PCT No.: PCT/NL2017/050824

§ 371 Date: Jun. 7, 2019

(30) Foreign Application Priority Data
Dec. 8, 2016 (NL) 2017954

(51) Int. Cl. A61B 17/29; A61B 90/00

(52) U.S. Cl. CPC A61B 17/29; A61B 90/03; A61B 2090/031; A61B 2017/294; A61B 2017/2912; A61B 2017/2903

(57) ABSTRACT

The invention relates to a surgical device for minimally invasive surgery. The surgical device comprises a shaft (10) extending along a longitudinal axis to a distal end thereof, and a surgical module (3) mounted to the distal end of the shaft and provided with a movable surgical element. The shaft includes a tube (7) that is rotatable relative to the shaft (10) and a further tube (5). The shaft further includes at least one slider (15A,B) connected to the surgical module and operatively driven by the rotatable tube via a rotary to linear linkage such that a rotation of the rotatable tube around the longitudinal axis induces a movement of the slider along the longitudinal axis thereby inducing a movement of the movable surgical element. The further tube is coaxial with the rotatable tube and rotationally stationary relative to the shaft. Further, the at least one slider is rotationally locked relative to the stationary tube.

The invention relates to a surgical device for minimally invasive surgery, comprising a shaft extending along a longitudinal axis to a distal end thereof, and a surgical module mounted to the distal end of the shaft and provided with a movable surgical element, wherein the shaft includes a tube that is rotatable relative to the shaft and a further tube, wherein the shaft further includes at least one slider connected to the surgical module and operatively driven by the rotatable tube via a rotary to linear linkage such that a rotation of the rotatable tube around the longitudinal axis induces a movement of the slider along the longitudinal axis thereby inducing a movement of the movable surgical element.

Typically, minimally invasive operations are performed through small portals for accessing deeper located tissue. During use of a known surgical device for minimally invasive surgery, the surgical module provided on the distal end of the shaft is brought into the body, via the portals, to manipulate said tissue. By interaction with the shaft, at a proximal end thereof, the at least one slider can be driven to induce a movement of the movable surgical element.

In the surgical device disclosed in International patent publication WO 2014/148898, one of the inventors being the inventor of the present invention, the rotary to linear linkage includes a three layer coaxial structure, viz. an inner tube and an outer tube as well as a pair of sliders provided with axially extending pins that traverse spiral shaped slits that are provided in both tubes. During operation, the inner and outer tube rotate in mutual reverse order thereby inducing the sliders to move in a direction parallel to the longitudinal axis of the tubes.

In principle, such multiple coaxial structures can be applied for moving the surgical elements of the surgical module, in order to meet minimally invasive surgery purposes. However, due to the small size of the portals, the total number of coaxial structures in the shaft for operating the surgical module is to be limited.

It is an object of the invention to provide a surgical device for minimally invasive surgery according to the preamble, wherein the number of coaxial structure in the shaft is reduced without loosing functionality of the surgical module. Thereto, according to the invention, the further tube, coaxial with the rotatable tube, is rotationally stationary relative to the shaft, wherein the at least one slider is rotationally locked relative to the stationary tube.

By rotationally locking the at least one slider to a stationary tube, the slider might be still movable in the longitudinal direction, when driven by the rotatable tube, while, on the other hand, the slider does not need to be located at the same longitudinal level of the both tubes, thereby enabling a two layer radial implementation of the rotation to linear linkage.

The invention is at least partly based on the insight that a rotation to linear linkage can in principle be realized by a two layer radial structure provided that the linear moving element is locked against rotational movement.

Advantageously, the stationary tube is provided, at its distal end, with a cut away extending along the longitudinal axis and receiving a proximal portion of the slider. Then, the stationary tube and the slider can be integrated in a single radial layer effectively reducing the number of radial layers of the shaft.

The invention also relates to a method of assembling a surgical device for minimally invasive surgery.

Further, the invention relates to a method for de-assembling a surgical device for minimally invasive surgery.

Other advantageous embodiments according to the invention are described in the following claims.

By way of example only, embodiments of the present invention will now be described with reference to the accompanying figures in which

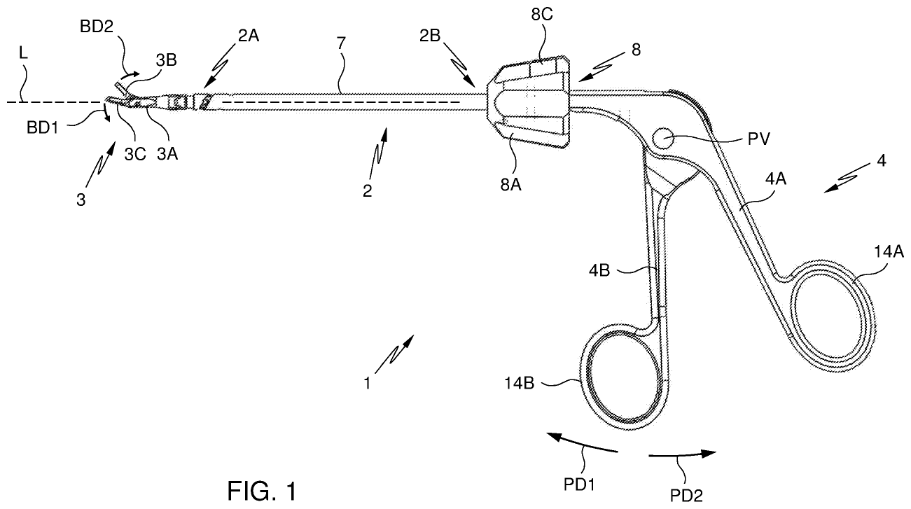

FIG. 1 shows a schematic view of a surgical device according to the invention in an assembled state;

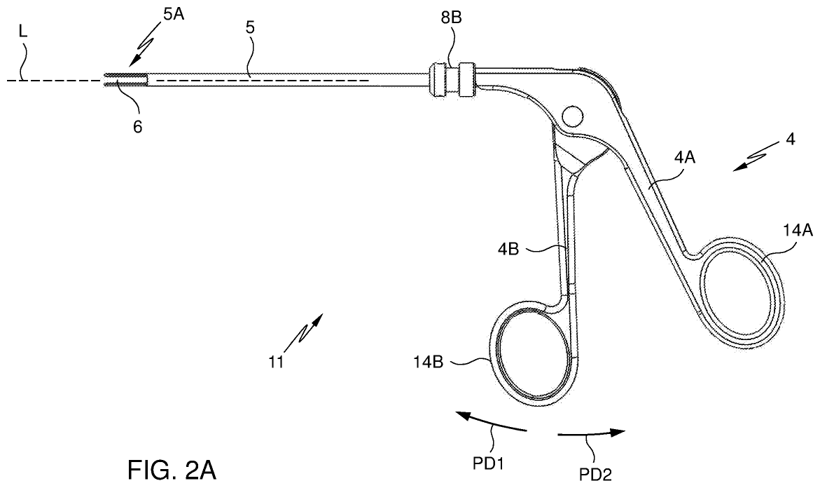

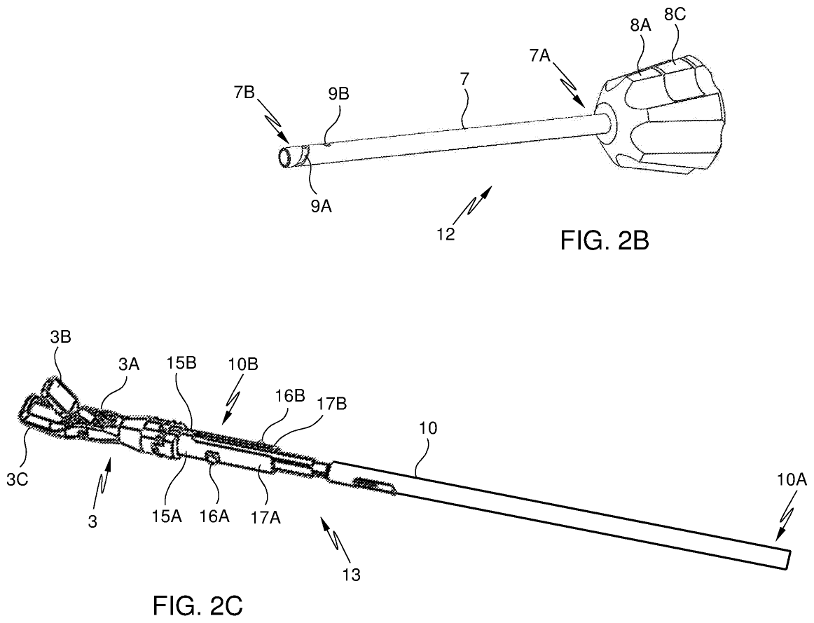

FIG. 2A shows a schematic view of a first unit of the surgical device shown in FIG. 1;

FIG. 2B shows a schematic perspective view of a second unit of the surgical device shown in FIG. 1;

FIG. 2C shows a schematic perspective view of a third unit of the surgical device shown in FIG. 1;

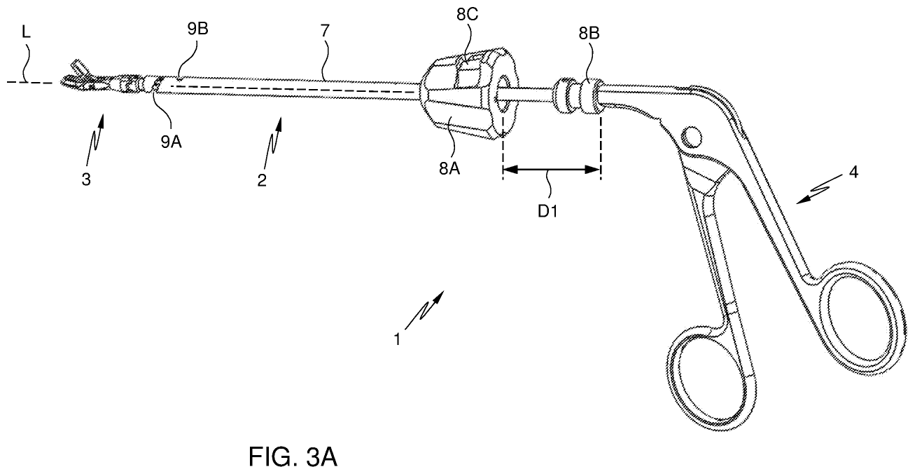

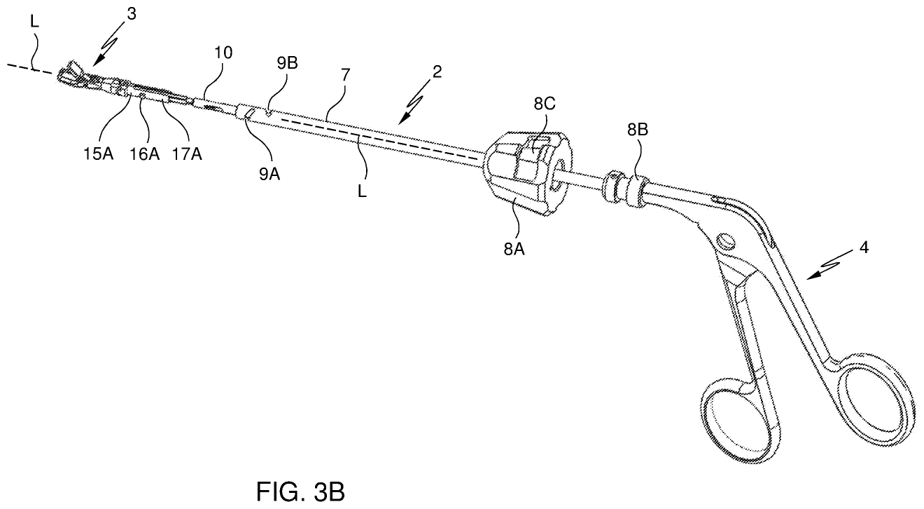

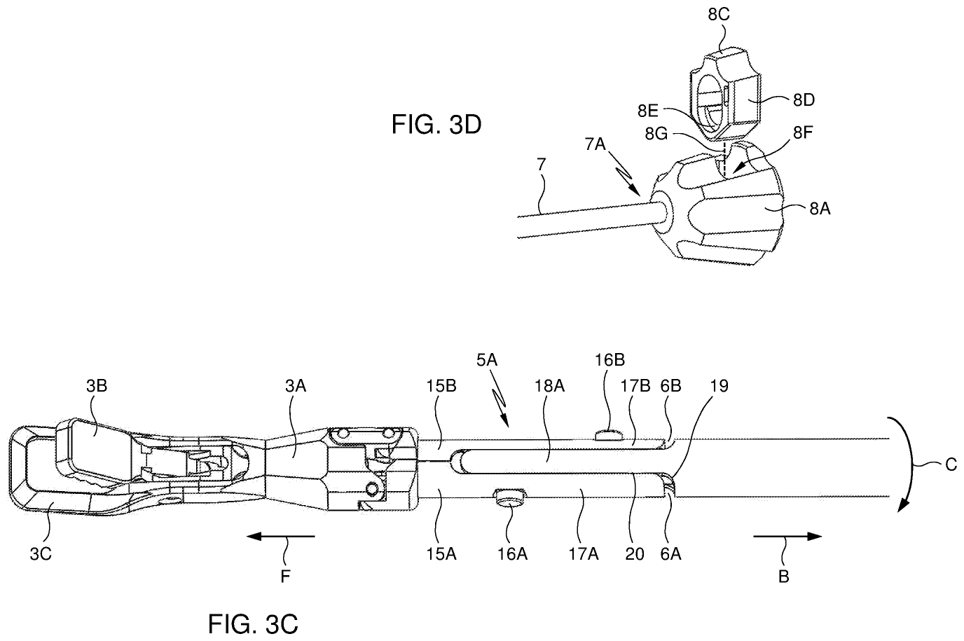

FIG. 3A shows a schematic perspective view of the surgical device shown in FIG. 1 wherein an actuation module is decoupled from a rotatable tube;

FIG. 3B shows a schematic perspective view of the surgical device shown in FIG. 3A wherein the third unit has been shifted away from the second unit;

FIG. 3C shows a schematic partial view of the third unit;

FIG. 3D shows a schematic partial view of the second unit;

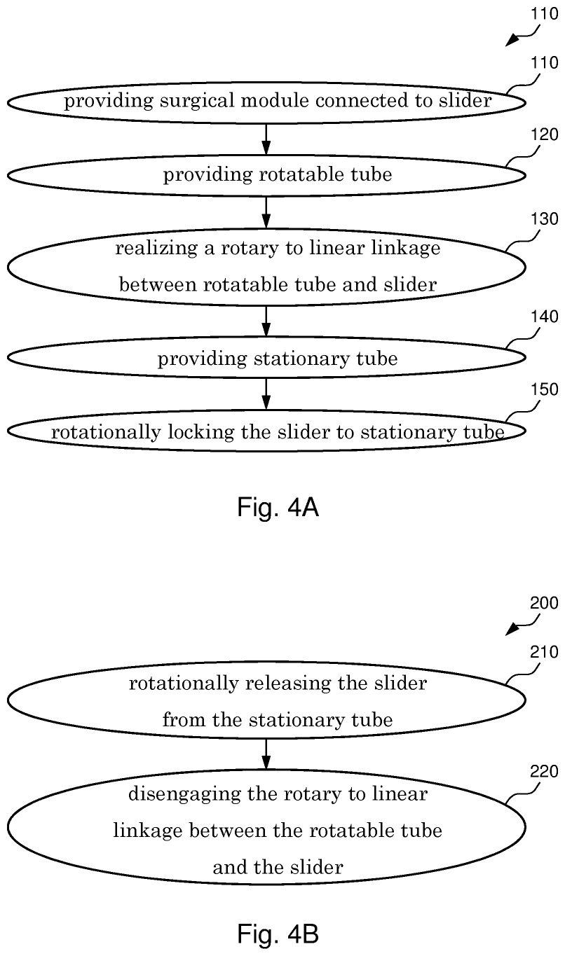

FIG. 4A shows a flow chart of an assembling method according to the invention, and

FIG. 4B shows a flow chart of a de-assembling method according to the invention.

The figures merely illustrate a preferred embodiment according to the invention. In the figures, the same reference numbers refer to equal or corresponding parts.

FIG. 1 shows a schematic perspective view of a surgical device 1 according to the invention in an assembled state. The device 1 is designed especially for use in minimally invasive surgery, and includes a shaft 2 extending along a longitudinal axis L from a proximal end 2A to a distal end 2B. Further, the device 1 includes a surgical module 3 that is mounted to the distal end 2B, and an actuation module 4 mounted to the proximal end 2A for handling the device 1.

The surgical module 3 includes a basic module 3A and two movable surgical elements, viz. a first and a second grasper beak 3B, 3C. The grasper beaks 3B, 3C are movable in a first swiveling direction BD1 and in a second swiveling direction BD2, reverse to the first swiveling direction BD1. The actuation module 4 includes a scissor type handling mechanism having a stationary portion 4A and a portion 4B that is pivotable in a first pivoting direction PD1 and in a second pivoting direction PD2, reverse to the first pivoting direction PD1, with respect to a pivoting axle PV on the stationary portion 4A. The stationary portion 4A and the pivotable portion 4B are each provided with finger grips 14A,B to facilitate manual operation.

The shaft 2 includes an outer tube 7 that is rotatable relative to the shaft 2 and an inner tube, not visible in FIG. 1, that is coaxial with the rotatable tube 7 and stationary relative to the shaft 2.

The surgical device 1 further includes a coupling unit having a first coupling element 8A rigidly connected to the rotating tube 7 and a second coupling element, not visible in FIG. 1, rotatably mounted on the stationary portion 4A of the actuation module 4. By actuating the actuation module 4 the rotatable tube 7 is brought into rotation, via the coupling unit 8, thereby moving the grasper beaks 3B, 3C, as explained below in more detail.

FIG. 2A shows a schematic view of a first unit 11 of the surgical device 1 shown in FIG. 1, in a de-assembled state.

The first unit 11 includes the actuation module 4 and the inner, stationary tube 5 indicated above. The stationary tube is fixedly connected to the actuation module 4. The stationary tube 5 is provided, at a distal end 5A thereof, with cut aways 6 for operational interaction with sliders as explained in more detail below. Further, the first unit 11 includes the second coupling element 8A mentioned above and rotatably mounted on the stationary portion 4A of the actuation module 4. The second coupling element 8A is rotatably driven by moving the pivotable portion 4B of the actuation module 4 in one of the pivoting directions PD1,2.

FIG. 2B shows a schematic perspective view of a second unit 12 of the surgical device 1 shown in FIG. 1, in the de-assembled state. The second unit 12 includes the rotatable tube 7 and the first coupling element 8A rigidly connected thereto, at a proximal end 7A of the rotatable tube 7. The rotatable tube 7 is provided, near its distal end 7B, with two spiral shaped slits 9A, 9B. Preferably, the spiral shape of the slits are mutually reversely oriented.

FIG. 2C shows a schematic perspective view of a third unit 13 of the surgical device 1 shown in FIG. 1, in the de-assembled state. The third unit 13 includes the surgical module 3 and an inner rod 10 connected, at its distal end 10B, to said surgical module 3. Further, the third unit 13 includes a pair of sliders 15A,B having a mainly elongate structure and arranged diametrically opposite to each other relative to the inner rod 10. The sliders 15A,B are connected, at a respective distal end thereof, with the surgical module 3 such that a movement of the respective slider 15A,B along the longitudinal axis L induces a corresponding grasper beak 3B,C swiveling movement in the first or second swiveling direction BD1, BD2. The sliders 15A,B are further each provided with a pin 16A,B extending radially outwardly to be received in a corresponding spiral shaped slit 9A,B on the rotatable tube 7 for forming a respective rotary to linear linkage. Then, in an assembled state of the surgical device 1, the sliders 15A,B are operatively driven via the rotary to linear linkages such that a rotation of the rotatable tube 7 around the longitudinal axis L induces a movement of the sliders 15A,B along the longitudinal axis L thereby inducing a movement of the grasper beaks 3B,C.

It is noted, generally, that the pin 16A,B can also be implemented as a bar preferably having a shape in conformity with the geometry of the corresponding spiral shaped slit 9A,B, thereby enlarging the contact area that the pin or bar 16A,B has in common with the corresponding slit, so that the rotary to linear linkage is firmer and/or has an improved wear resistivity.

In a process of assembling the surgical device 1, the second unit 12 and the third unit 13 are combined. In the combining step, a proximal end 10A is inserted into the distal end 7B of the rotatable tube 7 until the pins 16A,B are locked into the corresponding spiral shaped slits 9A,B, thus realizing a first rotary to linear linkage between the rotatable tube 7 and a first slider 15A, and a second rotary to linear linkage between the rotatable tube 7 and a second slider 15A. Then, a further combining step is performed wherein the first unit 11 is assembled by rotationally locking the sliders 15A,B relative to the stationary tube 5 such that a rotation of the rotatable tube 7 around the longitudinal axis L induces a movement of the sliders along the longitudinal axis L, in mutually opposite directions, thereby inducing a swiveling movement of the grasper beaks 3B,C in the first or second swiveling direction BD1, BD2. In the further combining step, the distal end 5A of the stationary tube 5 is inserted into the first coupling element 8a until a proximal portion 17A,B of the sliders 15A,B is received in the corresponding cut aways 6 of the stationary tube 5. Then, the first coupling element 8a rotatably engages with the second coupling element 8b in a rotational direction around the longitudinal axis L so that the actuation module 4 is driveably coupled to the rotatable tube 7 in a stable configuration. The sliders 15A,B can now still move in a longitudinal direction along the longitudinal axis L, but can not rotate relative to the stationary tube 5. Further, the inner rod 10 engages with the actuation module 4, the second coupling element 8b and/or the stationary tube 5 so that the inner rod 10 and the basic module 3A of the surgical module 3 is fixed along the longitudinal axis L.

FIG. 3A shows a schematic perspective view of the surgical device 1 wherein the actuation module 4 is decoupled from the rotatable tube 7. Here, the first and second coupling elements 8A,B of the coupling unit 8 are not coupled. In a process of de-assembling the surgical device 1, the sliders 15A,B are rotationally released from the stationary tube 5, i.e. the sliders are enabled to rotate with respect to the stationary tube 5. In the releasing step, the first and second coupling element 8A,B are disengaged so that the actuation module 4 and the rotatable tube 7 are not rotatably coupled anymore. Thereto, in the shown embodiment, the first coupling element 8A is provided with a release button 8C and a release mechanism releasing the first from the second coupling element 8A,B, e.g. by retracting an engaging finger into the first coupling element. Then, the first coupling element 8A shifts towards the distal end 5A of the stationary tube 5. By shifting the first coupling element 8A and the rotatable tube 7 relative to the stationary tube 5, also the sliders 15A,B move away from the stationary tube 5, from the cut aways 6 in the stationary tube 7 so that the sliders 15A,B can again freely rotate relative to said stationary tube 7. The first unit 11 can be disassembled.

In FIG. 3A, a state is shown wherein the actuation module 4 is decoupled from the rotatable tube 7. The first coupling element 8A has been shifted slightly along the longitudinal axis L, a first distance D1, towards the distal end 5A of the stationary tube 5.

FIG. 3C shows a schematic perspective view of the surgical device 1 in a further state wherein the third unit 13 has been shifted away from the second unit 12. Here, the third unit 13 including the surgical module 3 has been shifted away from the distal end 5A of the stationary tube 5, prior to final de-assembly of the first unit 11.

By shifting the third unit 13 away from the second unit 12, as a next step in the process of de-assembling, the surgical device 1, the rotary to linear linkages between the rotatable tube 7 on the one hand and the sliders 15A,B on the other hand are disengaged, e.g. by moving the radially outwardly extending pins 16A,B from the spiral shaped slits 9A,B radially inwardly, so that the sliders 15A,B can be removed from the rotating tube 7, thereby de-assembling the second unit 12 and the third unit 13. During operation of the assembled surgical device 1, the user may actuate the actuation module 4 by pivoting the pivotable portion 4B in a pivoting direction PD1, PD2 with respect to the pivoting axle PV on the stationary portion 4A, thereby rotating the rotatable tube 7 relative to the shaft 2. Via the rotary to linear linkages, the rotatable tube 7 drives the sliders 15A,B into a movement along the longitudinal axis L, in mutually opposite directions, thereby inducing a swiveling movement of the grasper beaks 3B,C in the first or second swiveling direction BD1, BD2, thereby opening or closing the beak of the grasper.

The shaft 2 includes the rotatable tube 7, the stationary shaft 5 and the sliders 15A,B, forming a radial multilayer configuration. By rotatably locking the sliders relative to the stationary tube 5, the sliders 14A,B and the stationary tube 5 can be designed such that they form a single radial layer and can be integrated to some extend, thereby advantageously reducing the total number of radial layers in the shaft 2. In the shown embodiment, the stationary tube 5 and the sliders 15A,B snugly fit inside the rotatable tube 7, so that the stationary tube 5 is an inner tube and the rotatable tube 7 is an outer tube. However, in principle, in another design, the stationary tube 5 is an outer tube while the rotatable tube 7 is an inner tube.

FIG. 3C shows a schematic partial perspective view of the third unit 13 of the surgical device 1. The detailed view shows said third unit 13 including the surgical module 3 being received by the distal end 5A of the stationary tube 5 provided with two cut aways or recesses 6A,B extending along the longitudinal axis L. In the shown embodiment, the cut aways 6A,B are arranged mutually diametrically with respect to the longitudinal axis L. However, in principle, the cut aways 6A,B can be arranged at another relative circumferential position, However, preferably, the cut aways are evenly distributed along the circumferential direction C. The distal end 5A of the stationary tube 5 forms locking members 18A,B between the cut aways 6A,B.

The number of cut aways 6A,B corresponds to the number of sliders 15A,B, in the shown embodiment two sliders 15A,B, viz. for receiving a proximal portion 17A,B of a corresponding slider 15A,B. The received sliders 15A,B may shift along the longitudinal axis L in a forward direction F and a backward direction B, but are locked by locking elements 18A,B against rotation relative to the stationary tube 5.

Preferably, the contour of the cut aways 6 corresponds to the dimensions of the sliders 15. In the shown embodiment, the contour 19 of the cut away is generally rectangular shaped corresponding to the mainly rectangular shaped edge 20 of the slider proximal portion 17A,B. By matching the geometry and/or dimensions and/or external contour of the cut aways to the corresponding geometry and/or dimensions and/or external contour of the slider proximal portion, the slider can smoothly slide along the longitudinal axis L while being locked against rotationally movement, i.e. a movement in the circumferential direction C relative to the stationary tube 5. In the shown embodiment, the sliders have a curved geometry matching with the bending contour of the stationary tube 5. Further, the edge of the locking elements 18A,B may have a beveled shape thereby locking the sliders to move racially inwardly. Then, the pins are locked in the corresponding slits 9A,B, in the assembled state.

Advantageously, the stationary tube 5 and the sliders 15A,B form a substantially closed ring, in a cross sectional view at the received proximal portion of the sliders 15A,B, transverse to the longitudinal axis L. The ring is alternatingly formed by locking elements and sliders, respectively, thereby integrally forming a single radial layer in the radial multilayer structure of the shaft 2.

It is noted that the sliders 15 can be rotationally locked relative to the stationary tube 5 in another manner, e.g. by shaping the sliders and the distal end of the stationary tube 5 such that the sliders receive a single or a multiple number of longitudinally extending portions of the stationary tube.

It is further noted that, in principle, a rotary to linear linkage can be realized in a reverse constellation, i.e. by providing a pin radially extending from a rotatable tube into a spiral shaped slit provided in a slider. Further, the slider can be racially exterior to the rotatable tube. Further, the rotary to linear linkage can be implemented using other technical principles, e.g. using a screw linkage.

In the shown embodiment, the pivotable portion 4B of the actuation module 4 forms an actuation element driving the rotatable tube via the coupling unit 8. If desired, the actuation element can be designed in a different way, e.g. as a member that is movable along a linear, curved or straight path.

FIG. 3D shows a schematic partial view of the second unit 12 provided, at its proximal end 7A, with the first coupling element 8A. The first coupling element 8A is provided with a generally block shaped release element 8D having an upper surface 8C acting as release button when the release element 8D is received in a cavity 8F of the first coupling element 8A such that it can merely move along a path 8G racially extending from the longitudinal axis L of the shaft 2. In the shown embodiment, the release element 8D has a through hole with a threshold profile 8E cooperating with the second coupling element 8B. During use of the surgical device 1, the release element 8D is received in the cavity 8F of the first coupling element 8A, so that the first and second coupling elements 8A, 8B are coupled. Here, the second coupling element 8B is received in the through hole of the release element 8D, the second coupling element 8B engaging the threshold profile 8E. By pressing the release element 8D, the first and second coupling elements 8A, 8B are decoupled. Then, the release element 8D including the threshold profile 8E advances further into the cavity 8F of the first coupling element, thereby releasing the second coupling element 8B from the threshold profile 8E enabling the second coupling element 8B to freely move along the longitudinal axis L of the shaft 2. Advantageously, the coupling unit 8 can further be arranged for automatically retracting the release element 8D thereby decoupling the first coupling element 8A from the second coupling element 8B if a force exerted on the rotary to linear linkage exceeds a predetermined level. Then, the second coupling element 8B forces the release element 8D further into the cavity 8F of the first coupling element. To this end, the threshold profile 8E is not perfectly oriented transverse to the longitudinal axis L of the shaft, but slightly curved and/or tilted relative to a contact surface of the second coupling element 8B facing the threshold profile 8E, thus serving as a self-releasing coupling, preferably in a reversible manner, facilitating a transfer of forces from the second coupling element 8B towards the release element 8D received in the cavity 8F of the first coupling element 8A if said forces are below a predetermined level. Again, after movement of the release element 8D into the cavity 8F, the second coupling element 8B and the threshold profile 8E do not engage each other anymore, enabling the second coupling element 8B to freely move along the longitudinal axis L of the shaft, through the through hole of the release element 8D. Upon moving into the cavity 8F, the release element 8D including the upper surface 8C acting as release button is situated in a depressed position, thus providing a clear visible indication to the user that the actuation module 4 has decoupled. In principle, the first coupling element 8A can be coupled again to the second coupling element 8B, e.g. by facilitating a reverse movement of the release element 8D back to its earlier position in the cavity 8F engaging with the second coupling element 8B. actuation module can be coupled to

In another embodiment, the coupling unit 8 has a limited torque transfer for transferring a torque from the first coupling element 8a driven by the actuation element 4B to the second coupling element 8b driving the rotatable tube 7. By limiting the torque that is transferred by the coupling unit, the undesired occurrence of material fatigue or even breakage of surgical elements can be counteracted. Further, an undesired overload in force exerted by surgical elements on tissue can be counteracted. The coupling unit 8 having a limited torque transfer can e.g. be implemented using a friction coupling element. Further, the coupling unit 8 can be arranged for releasing the actuation element from the rotatable tube 7 if a force exerted on the rotary to linear linkage exceeds a predetermined level, for safety purposes.

Preferably, the surgical device is demountable as described above. However, in principle, the surgical device can also be formed as an integral unit.

The pitch of the spiral shaped slits 9A,B can be uniform, i.e. constant along its bending profile. The value of the pitch can be selected between a relatively small value so that the corresponding pin moves relatively small when rotating the tube, and a relatively large value so that the corresponding pin moves relatively quickly when rotating the tube. Further, the pitch of the spiral shaped slit can be non-uniform, i.e. varying as a function of the circumferential position of the slit. As a consequence, also the speed of the pin varies when rotating the tube with a constant rotation speed. As an example, the pitch of the slit may decrease at an end portion of the slit such that the speed of the corresponding pin reduces when reaching an end of its guiding path.

In the described embodiments, the surgical module 1 includes two movable surgical elements, viz. two beaks 3B,C, wherein the shaft 2 includes two sliders 15A,B forming a pair of sliders connected to the surgical module 3, each of the sliders 15A,B being operatively driven by the rotatable tube 7 via a respective rotary to linear linkage such that a rotation of the rotatable tube 7 around the longitudinal axis L induces a movement of both sliders 15A,B along the longitudinal axis L thereby inducing a movement of both movable surgical elements 3B,C. In the shown embodiment, the sliders 15A,B move in opposite directions. However, in principle, the sliders 15A,B may also move in the same direction, along the longitudinal axis L. Further, the surgical module 1 may include another number of sliders, e.g. a single slider, three or four sliders, for moving a corresponding number of surgical elements on the surgical module 3, i.e. a single surgical element or three or four surgical elements, respectively. Then, also the number of cut aways 6 in the stationary tube 5 corresponds to the number of sliders 15, each of the cut aways 6 receiving a proximal portion of a corresponding slider 15.

The surgical module 4 can be implemented as a grasper or another surgical module, e.g. a cutter module.

FIG. 4A shows a flow chart of a method 100 of assembling a surgical device for minimally invasive surgery, comprising a shaft extending along a longitudinal axis to a distal end thereof. The method comprises a step of providing 110 a surgical module provided with a movable surgical element connected to a slider, a step of providing 120 a tube that is rotatable relative to the shaft, a step of realizing 130 a rotary to linear linkage between the rotatable tube and the slider for operationally driving the slider by the rotatable tube, a step of providing 140 a tube that is coaxial with the rotatable tube and rotationally stationary relative to the shaft, and a step of rotationally locking 150 the slider relative to the stationary tube such that a rotation of the rotatable tube around the longitudinal axis induces a movement of the slider along the longitudinal axis thereby inducing a movement of the movable surgical element.

Advantageously, the step of realizing a rotary to linear linkage includes inserting a pin radially extending from the slider or rotatable tube into a spiral shaped slit provided in the rotatable tube or slider, respectively.

Further, the step of locking the slider may include receiving a proximal portion of the slider into a cut away extending along the longitudinal axis and provided in the stationary tube, at its distal end.

FIG. 4B shows a flow chart of a method 200 of de-assembling a surgical device for minimally invasive surgery, comprising a step of rotationally releasing 210 the slider from the stationary tube, and a step of disengaging 220 the rotary to linear linkage between the rotatable tube and the slider.

The invention is not restricted to the embodiments described herein. It will be understood that many variants are possible.

It is noted, as an example, that the shaft of the surgical device may include a further rotatable tube that is coaxial with the stationary tube, and a further at least one slider connected to the surgical module and operatively driven by the further rotatable tube via a further rotary to linear linkage such that a rotation of the further rotatable tube around the longitudinal axis induces a movement of the slider along the longitudinal axis thereby inducing a further movement of the surgical module, wherein the further at least one slider is rotationally locked relative to the stationary tube, thereby including two rotatable tubes that may, in dependently of each other, drive corresponding sliders for inducing movements of the surgical unit, e.g. in multiple degrees of freedom. In an embodiment, the cut aways corresponding to a first set of sliders driven by a first rotatable tube may be provided in a first longitudinal regime at the distal end of the stationary tube, while the cut aways corresponding to a second set of sliders driven by a second rotatable tube may be provided in a second longitudinal regime at the distal end of the stationary tube, staggered from the first longitudinal regime relative to the longitudinal axis.

These and other embodiments will be apparent for the person skilled in the art and are considered to fall within the scope of the invention as defined in the following claims. For the purpose of clarity and a concise description features are described herein as part of the same or separate embodiments. However, it will be appreciated that the scope of the invention may include embodiments having combinations of all or some of the features described.

1. A surgical device for minimally invasive surgery, comprising a shaft extending along a longitudinal axis to a distal end thereof, and a surgical module mounted to the distal end of the shaft and provided with a movable surgical element, wherein the shaft includes a tube that is rotatable relative to the shaft and a further tube that is coaxial with the rotatable tube, wherein the shaft further includes at least one slider connected to the surgical module and operatively driven by the rotatable tube via a rotary to linear linkage such that a rotation of the rotatable tube around the longitudinal axis induces a movement of the slider along the longitudinal axis thereby inducing a movement of the movable surgical element, wherein the further tube, coaxial with the rotatable tube, is rotationally stationary relative to the shaft, and wherein the at least one slider is rotationally locked relative to the stationary tube.

2. The surgical device according to claim 1, wherein the stationary tube is provided, at its distal end, with a cut away extending along the longitudinal axis and receiving a proximal portion of the slider.

3. The surgical device according to claim 2, wherein the contour of the cut away corresponds to the dimensions of the slider.

4. The surgical device according to claim 2, wherein, in a cross sectional view at the received proximal portion of the slider, the stationary tube and the slider form a substantially closed ring.

5. The surgical device according to claim 1, wherein the stationary tube and the slider snugly fit inside the rotatable tube.

6. The surgical device according to claim 1, wherein the rotary to linear linkage includes a pin or bar radially extending from the slider or rotatable tube into a spiral shaped slit provided in the rotatable tube or slider, respectively.

7. The surgical device according to claim 1, wherein the pitch of the spiral shaped slit is non-uniform.

8. The surgical device according to claim 1, wherein the surgical module includes two movable surgical elements and wherein the shaft includes a pair of sliders connected to the surgical module, each of the sliders being operatively driven by the rotatable tube via a respective rotary to linear linkage such that a rotation of the rotatable tube around the longitudinal axis induces a movement of both sliders along the longitudinal axis thereby inducing a movement of both movable surgical elements.

9. The surgical device according to claim 1, wherein the shaft includes a further rotatable tube that is coaxial with the stationary tube, and a further at least one slider connected to the surgical module and operatively driven by the further rotatable tube via a further rotary to linear linkage such that a rotation of the further rotatable tube around the longitudinal axis induces a movement of the slider along the longitudinal axis thereby inducing a further movement of the surgical module, wherein the further at least one slider is rotationally locked relative to the stationary tube.

10. The surgical device according to claim 1, further comprising an actuation element that is rotatably coupled to the rotatable tube, at a proximal end of the shaft, via a coupling unit that is arranged for releasing the actuation element from the rotatable tube if an exerted force on the rotary to linear linkage exceeds a predetermined level.

11. The surgical device according to claim 1, further comprising an actuation element that is rotatably coupled to the rotatable tube, at a proximal end of the shaft, via a coupling unit having a limited torque transfer.

12. The surgical device according to claim 10, wherein the coupling unit is provided with a release button that is arranged for releasing the actuation element upon pressing the button.

13. A method of assembling a surgical device for minimally invasive surgery, comprising a shaft extending along a longitudinal axis to a distal end thereof, the method comprising the steps of:
providing a surgical module provided with a movable surgical element connected to a slider;
providing a tube that is rotatable relative to the shaft;
realizing a rotary to linear linkage between the rotatable tube and the slider for operationally driving the slider by the rotatable tube;
providing a tube that is coaxial with the rotatable tube and rotationally stationary relative to the shaft, and
rotationally locking the slider relative to the stationary tube such that a rotation of the rotatable tube around the longitudinal axis induces a movement of the slider along the longitudinal axis thereby inducing a movement of the movable surgical element.

14. The method according to claim 13, wherein the step of realizing a rotary to linear linkage includes inserting a pin or bar radially extending from the slider or rotatable tube into a spiral shaped slit provided in the rotatable tube or slider, respectively.

15. The method according to claim 13, wherein the step of locking the slider includes receiving a proximal portion of the slider into a cut away extending along the longitudinal axis and provided in the stationary tube, at its distal end.

16. A method of de-assembling a surgical device for minimally invasive surgery according to claim 1, comprising the steps of:
rotationally releasing the slider from the stationary tube, and
disengaging the rotary to linear linkage between the rotatable tube and the slider.
